SEQUENCE LISTING (20 sequences)

SEQ ID NO: 1 - DNA, 23 bp, Artificial Sequence
  L32-Forward Primer
cacaatgtcaaggagctggaagt

SEQ ID NO: 2 - DNA, 22 bp, Artificial Sequence
  L32-Reverse Primer
tctacaatggcttttcggttct

SEQ ID NO: 3 - DNA, 22 bp, Artificial Sequence
  OPN-Forward Primer
agcaagaaactcttccaagcaa

SEQ ID NO: 4 - DNA, 23 bp, Artificial Sequence
  OPN-Reverse Primer
gtgagattcgtcagattcatccg

SEQ ID NO: 5 - DNA, 21 bp, Artificial Sequence
  OCN-Forward Primer
ctgacctcacagatgccaagc

SEQ ID NO: 6 - DNA, 22 bp, Artificial Sequence
  OCN-Reverse Primer
tggtctgatagctcgtcacaag

SEQ ID NO: 7 - DNA, 20 bp, Artificial Sequence
  BSP-Forward Primer
cagggaggcagtgactcttc

SEQ ID NO: 8 - DNA, 20 bp, Artificial Sequence
  BSP-Reverse Primer
agtgtggaaagtgtggcgtt

SEQ ID NO: 9 - DNA, 21 bp, Artificial Sequence
  ALP-Forward Primer
ccaactcttttgtgccagaga

SEQ ID NO: 10 - DNA, 23 bp, Artificial Sequence
  ALP-Reverse Primer
ggctacattggtgttgagctttt

SEQ ID NO: 11 - DNA, 19 bp, Artificial Sequence
  ColIalphaI-Forward Primer
gctcctcttaggggccact

SEQ ID NO: 12 - DNA, 19 bp, Artificial Sequence
  ColIalphaI-Reverse Primer
ccacgtctcaccattgggg

SEQ ID NO: 13 - DNA, 21 bp, Artificial Sequence
  Runx2-Forward Primer
tgttctctgatcgcctcagtg

SEQ ID NO: 14 - DNA, 23 bp, Artificial Sequence
  Runx2-Reverse Primer
cctgggatctgtaatctgactct

SEQ ID NO: 15 - DNA, 19 bp, Artificial Sequence
  OSX-Forward Primer
atggcgtcctctctgcttg

SEQ ID NO: 16 - DNA, 21 bp, Artificial Sequence
  OSX-Reverse Primer
tgaaaggtcagcgtatggctt

SEQ ID NO: 17 - DNA, 23 bp, Artificial Sequence
  PPARgamma -Forward Primer
tgaaactctgggagattctcctg

SEQ ID NO: 18 - DNA, 24 bp, Artificial Sequence
  PPARgamma -Reverse Primer
ccatggtaatttcttgtgaagtgc

SEQ ID NO: 19 - DNA, 22 bp, Artificial Sequence
  Aggrecan-Forward Primer
gcgtgagcatccctcaaccatc

SEQ ID NO: 20 - DNA, 22 bp, Artificial Sequence
  Aggrecan-Reverse Primer
ggcagtggtcacaggatgcatg

(19) United States
(12) Patent Application Publication
(10) Pub. No.: US 2010/0150880 A1
(43) Pub. Date: Jun. 17, 2010

(54) Isolated mesenchymal cell population and method for isolating and using same

(75) Inventors: Jane E. Aubin, Toronto (CA); Shousaku Itoh, Toyonaka (JP)

Correspondence Address: SEED INTELLECTUAL PROPERTY LAW GROUP PLLC, 701 FIFTH AVE, SUITE 5400, SEATTLE, WA, 98104, US

(73) Assignee: The Governing Council of the University of Toronto, Toronto (CA)

(21) Appl. No.: 12/590,323

(22) Filed: Nov. 4, 2009

Related U.S. Application Data
(60) Provisional application No. 61/111,221, filed on Nov. 4, 2008.

(51) Int. Cl. A61K 35/28; C12N 5/07; C12N 5/071

(52) U.S. Cl. 424/93.7; 435/354; 435/372

(57) ABSTRACT

The present invention provides a novel isolated mesenchymal cell population of highly purified osteoprogenitors (HipOPs) that can be used in the formation of bone tissue and methods for isolating and using same.

### RELATED APPLICATIONS

This application claims the benefit of priority of U.S. provisional application No. 61/111,221, filed Nov. 4, 2008, entitled “Isolated Mesenchymal Cell Population and Method for Isolation and Using Same” which is incorporated by reference herein.

### FIELD OF THE INVENTION

The invention relates generally to a novel isolated population of mesenchymal cells, compositions comprising same and to a method for isolating, identifying and using same.

### BACKGROUND OF THE INVENTION

As established originally by Freidenstein, at least some cells within bone marrow stromal populations are multipotential and can differentiate in vitro into osteoblasts, chondrocytes, adipocytes and myoblasts, which has led to the population frequently being designated mesenchymal stem cells, multipotential marrow stromal cells (MSCs) or skeletal stem cells (SSCs) amongst other names^{1-10 11}. MSCs from human and rat bone marrow have been the most extensively characterized, because they are relatively easy to isolate by their phenotype of adherence to plastic and extensive expansion capacity in culture. In contrast, murine MSCs are far more difficult both to isolate from bone marrow and to expand in culture⁷, at least in part due to significant contamination by hematopoietic cells that are also directly adherent to plastic and bind to MSCs via adhesion molecules, cytokine receptors, and extra cellular matrix proteins¹²⁻¹⁵. The hematopoietic cells persist in these cultures even after serial passage due to the ability of stromal cells to support granulopoiesis and B cell lymphopoiesis even in the absence of exogenous growth factors and cytokines^{7,16}. Thus, unambiguous characterization of the intrinsic phenotypic and functional properties of murine MSCs requires depletion of lineage committed hematopoietic cells from mouse bone marrow cultures.

Others have described isolation of MSCs from murine bone marrow by negative selection, such as Baddoo et al. (Journal of Cellular Biochemistry 89:1235-1249 (2003)), however Baddo selected against CD34+ cells, thus removing certain MSC types and they did not identify any “niche cells” nor did their cells have the ability to form osteoblasts or donor bone marrow upon transplantation.

U.S. Pat. No. 7,303,769 B2 describes the isolation of pluri-differentiated mesenchymal progenitor cells but again do not describe niche markers nor in their transplantation experiments show that their cells upon transplantation can form an organ structure or bone of donor origin. The bone structure comprises those elements typically found in normal bone with cortical and trabecular bone tissue, bone cells (osteoblasts, osteocytes, osteoclasts), bone marrow and vascular tissue.

Thus there is need for a method for isolating MSCs and for a cell population that can form osteoblasts and whole bone structure both in vitro and upon transplantation.

### SUMMARY OF THE INVENTION

In one embodiment the invention provides a method for purifying mesenchymal cells comprising the steps of:

(a) providing or obtaining bone marrow cells from a subject, such as mouse or human;

(b) expanding the cells

(c) negatively selecting cells, for example fractionating the cells using negative selection for certain markers, for instance:
(i) for mouse mesenchymal cells, negatively selecting for one or more of the following markers, but in one embodiment all of the markers: CD5, CD45, CD11b, Gr-1, 7-4, Ter-119 and CD45R; and
(ii) for human mesenchymal cells, negatively selecting for one or more of the following markers, but in one embodiment all of the markers: CD2, CD3, CD11b, CD14, CD15, CD16, CD19, CD45, CD56, CD123, and CD235a;

(d) collecting the fraction of mesenchymal progenitor cells.

In one embodiment the negative selection is conducted using markers conjugated microbeads selected from one or more of, or in one embodiment all of, the following markers:
(i) for mouse mesenchymal cells: CD5, CD45, CD11b, Gr-1, 7-4, Ter-119 and CD45R; and
(ii) for human mesenchymal cells: CD2, CD3, CD11b, CD14, CD15, CD16, CD19, CD45, CD56, CD123, and CD235a;

In one embodiment the cells that do not adhere to said microbeads are collected.

In one embodiment the cells prior to or after expansion are treated to obtain a cell suspension.

In another embodiment the cellular aggregates are removed from the cell suspension.

In another embodiment, disclosed herein is method for isolating mesenchymal stem cells comprising:

(a) obtaining bone marrow cells, in one embodiment from a subject or bone sample or other source.

(b) treating the cells to obtain a cell suspension, in one embodiment the cell aggregates are removed from the cell suspension;

(c) expanding the bone marrow cells,

(d) sorting and or fractionating cells with desired markers and selecting for same. In one embodiment the cells were sorted using microbeads, in one embodiment, where the cells are mouse cells CD5 conjugated microbeads. In another embodiment where the cells are mouse cells, CD45R, CD11b, Gr-1, 7-4, Ter-119 and/or CD45 conjugated microbeads can also be used. In yet another embodiment where the cells are human cells, CD2, CD3, CD11b, CD14, CD15, CD16, C19, CD45, CD56, CD123, and/or CD235a conjugated microbeads can be used. In another embodiment, all of the above-noted markers for the respective cell types are used.

In another embodiment the cells were plated on alpha-MEM medium supplemented with antibiotics and 10% FCS. In another embodiment, non-adherent cells were removed by washing 3 times with PBS and then after about two weeks (or 14 days), the cells had expanded to subconfluent and were detached with trypsin-EDTA solution (0.2% trypsin, 1 mM EDTA).

In one embodiment, the cells can be murine cells. In another embodiment the cells are human cells.

In one embodiment the isolated cells were highly purified osteoprogenitor cells (HipOps).

In one embodiment of the method disclosed herein, the cells are expanded to subconfluent. In one embodiment the cells are expanded for about 14 days.

In one embodiment the invention provides a population of mesenchymal stem cells isolated using the method of the invention.

In one embodiment the invention provides a composition comprising a population of mesenchymal stem cells isolated using the method of the invention.

In another embodiment, the invention provides a population of mesenchymal stem cells, such as isolated mesenchymal stem cells, that have CD90, CD73, CD44, CD105 and/or Sca-1 markers. In one embodiment the stem cells have CD 73 and CD90 cell surface markers. In another embodiment said markers are present in the cell population in the following amounts CD90 (about 26.4%), CD73 (about 43.0%), CD44 (about 36.9%), CD105 (about 62.7%), and Sca-1 (about 98.3%). In one embodiment, the population of mesenchymal stem cells has cells that are CD34+. In another embodiment, the mesenchymal stem cells are Sca-1+. In another embodiment the mesenchymal stem cell population of the invention has cells that comprise niche markers. In one embodiment the niche markers are selected from the group consisting of: Angiopoietin1, N-cadherin, PTH1R, Jagged 1, and CXCL12. In one embodiment said mesenchymal cells are derived from murine cells.

In another embodiment, the mesenchymal stem cell population of the present invention can differentiate into osteoblasts, adipocytes and/or chondrocytes under appropriate culture conditions, such as the respective cell differentiating inducing conditions, for example cultured with the appropriate inducing medium, such as osteogenic, adipogenic and chondrogenic induction medium respectively. In one embodiment, the appropriate culture conditions and appropriate inducing media are those specified in the Examples, for instance in the “Materials and Methods section or Example 2 herein. In another embodiment, examples of suitable conditions and media are disclosed in Aubin J E. Osteoprogenitor cell frequency in rat bone marrow stromal populations: role for heterotypic cell-cell interactions in osteoblast differentiation. J Cell Biochem. (1999) 72(3):396-410 (for osteogenesis); Falconi D, Oizumi K, Aubin J E. Leukemia inhibitory factor influences the fate choice of mesenchymal progenitor cells. Stem Cells. (2007) 25(2):305-312 (for adipogenesis); and Zhang S, Uchida S, Inoue T, Chan M, Mockler E, Aubin J E. Side population (SP) cells isolated from fetal rat calvaria are enriched for bone, cartilage, adipose tissue and neural progenitors. Bone. (2006) 38(5):662-670 (for chondrogenesis).

Appropriate culture conditions and appropriate inducing media would be known to those of skill in the art after in reading this specification.

In another aspect, the invention provides a population of isolated mesenchymal stem cells that can form bone tissue, including cortical, trabecular bone, fat tissue and vascular system tissue. In one embodiment, the cell population of the present invention can form bone tissue in vitro and in another in vivo upon transplantation.

In one embodiment the isolated mesenchymal stem cell population of the invention can be used in the prevention or treatment of conditions where generation of bone tissue is beneficial, for instance, osteoporosis, bone fracture treatment, such as regenerative medicine, i.e., ex vivo bone engraftment in the field of orthopaedic or dental surgery.

In another aspect of the invention, the isolated mesenchymal stem cell population can be murine or human mesenchymal stem cells and the method of the invention can be used to isolate same.

In one aspect of the invention, the invention provides a kit for conducting the method of the invention comprising one or more of the following: culture medium, microbeads for sorting, and instructions for using same. In another embodiment the invention can provide a kit comprising a sample of isolated mesenchymal stem cells of the present invention and optionally culture medium and or instructions for use in experiments and/or in transplantation.

Other features and advantages of the present invention will become apparent from the following detailed description and accompanying drawings. It should be understood, however, while the detailed description and the specific examples may indicate preferred embodiments of the invention, they are given by way of illustration only, since various changes and modifications within the spirit and scope of the invention will become apparent to those skilled in the art from this detailed description.

### BRIEF DESCRIPTION OF THE DRAWINGS

The invention will be better understood with reference to the drawings which are used for illustrative purposes only and are not meant to limit the scope of the invention.

FIG. 1 is a schematic diagram illustrating the method of isolating the HipOPs population of the present invention and as further described in Example 1.

FIG. 2 is a FACS analysis as described in Example 1. (a) Bone Marrow Stromal Cells (BMSCs) at the days indicated and sorted HipOPs were stained with APC-anti-Sca-1 Abs. Graphs are displayed with forward scatter (FSC; x-axis) and Sca-1 (y-axis). (b) BMSCs and HipOPs were stained with the panel of typical “mesenchymal stem cell markers”.

FIG. 3 show stains illustrating the presence of various cell types that form when BMSCs or HipOPs are cutured under conditions supporting differentiation. (CFU-O, CFU-ALP and ALP+Mineral+cells) CFU-O, CFU-ALP and ALP+Mineral+cells in BMSCs or HipOPs cultured in osteogenic induction medium as described in Example 2. After culture with osteogenic condition medium for 4 weeks, cells were double stained for ALP and mineral deposition (von Kossa), and were re-stained with methylene blue. (Oil-Red-O) Cells were cultured in adipogenic induction medium 1 week. Adipocyte colonies were identified by Oil-Red-O staining. (Type 2 collagen) Cells were cultured in chondrogenic induction medium for 2 weeks. Cells were stained with anti-type 2 collagen antibody and a Vectastain Elite ABC kit, and re-stained with methylene blue.

FIG. 4 are graphs of the limiting dilution analysis as described in Example 2.

FIG. 5A are graphs illustrating mRNA transcription levels of the various markers indicated for mouse BMSCs and HipOPs at day 0 as described in Example 2. Values are expressed as means±S.D. Asterisks indicate statistically significant differences: p<0.05 (*), p<0.01 (**), p<0.005 (***).

FIG. 5B are graphs illustrating the relative mRNA transcription levels of various mesenchymal cell differentiation marker genes in BMSCs (clear bars) and HipOPs (black bars) cultured with osteogenic induction medium (OPN, OCN, BSP, ALP, Col1α1, Runx2, OSX, Ang1, N-cadherin, PTH1R, Jag1, and CXCL12), adipogenic induction medium (PPARγ), or chondrogenic induction medium (Aggrecan) as described in Example 2. Values are expressed as means±S.D. Asterisks indicate statistically significant differences: p<0.05 (*), p<0.01 (**), p<0.005 (***)

FIG. 6 illustrates the results of Example 3 where it is shown that mouse HipOPs can form a complete skeletal organ after transplantation. (a) MicroCT 3-D reconstructions of a typical transplant of BMSCs at 8 weeks after transplantation. Bar=1 mm. (b) MicroCT 3-D reconstructions of a typical transplant of HipOPs at 8 weeks after transplantation. Bar=1 mm. (c) The comparison of volume of total mineralized tissue between BMSCs and HipOPs. Data are expressed as means of three independent experiments±S.D. Asterisks indicate statistically significant differences: p<0.005 (***). (d-g) Histology of transplants of HipOPs harvested at 8 weeks. Cb, cortical bone; Tb, trabecular bone; ob, osteoblast; oc, osteoclast; ad, adipose tissue; hem, hematopoietic cells, H&E. d×100: e-g×400. Bars=50 μm (d): Bars=30 μm (e-g). (h-k) Frozen sections of transplants of HipOPs were stained with anti-H-2K^{b} Abs and re-stained with fast green. sin, sinusoid; os, osteocyte. h ×100: i-k ×400. Bars=50 μm (h): Bars=30 μm (i-k).

FIG. 7 (A) is a graph illustrating the amount of bone formed in transplants seeded with unfractionated human bone marrow stromal cells (hBMSCs) or with human HipOPs (hHipOPs) as a comparison of volume of total mineralized tissue between hBMSCs and hHipOPs. Data are from two independent experiments; the symbols indicate the values from individual experiments and the line marks the average of the two results. Approximately 10× more bone was formed in transplants seeded with hHipOPs than in transplants seeded with hBMSCs. (B) Histology of transplants of hHipOPs harvested at 8 weeks, trabecular bone (Tb).

### DEFINITIONS

Unless defined otherwise, all technical and scientific terms used herein have the same meaning as commonly understood by one of ordinary skill in the art to which this invention belongs.

“Mesenchymal stem cells” (“MSCs”) as used herein are multipotent stem cells that can differentiate into a variety of cell types. Cell types that MSCs have been shown to differentiate into in vitro or in vivo include osteoblasts, chondrocytes, myocytes, and adipocytes.

“BMSC” as used herein are bone marrow stromal cells.

“Isolated MSC population” or “Isolated MSC” as used herein is an isolated heterogeneous MSC population.

“HipOps” as used herein refers to the highly purified population of osteoprogenitor mesenchymal stem cells isolated and identified in the present invention.

“Niche Marker” as used herein refers to Ang1, N-cadherin, PTH1R, Jag1, and CXCL12.

“Niche Cells” as used herein refers to osteoblasts and cells supporting hematopoietic stem cells.

As used herein, the terms “comprising,” “including,” and “such as” are used in their open and non-limiting sense.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” The term “about” means plus or minus 0.1 to 50%, 5-50%, or 10-40%, preferably 10-20%, more preferably 10% or 15%, of the number to which reference is being made.

Further, it is to be understood that “a,” “an,” and “the” include plural referents unless the content clearly dictates otherwise. For example, reference to “a compound” includes a mixture of two or more compounds.

The terms “administering” and “administration” refer to the process by which a therapeutically effective amount of a compound or cells or a composition contemplated herein is delivered to a subject for prevention and/or treatment purposes. Compositions are administered in accordance with good medical practices taking into account the subject's clinical condition, the site and method of administration, dosage, patient age, sex, body weight, and other factors known to physicians.

The terms “subject”, “individual” or “patient” refer to an animal including a warm-blooded animal such as a mammal, which is afflicted with or suspected of having or being pre-disposed to a condition and/or disease as disclosed herein. Preferably, the terms refer to a human. The terms also include domestic animals bred for food, sport, or as pets, including horses, cows, sheep, poultry, fish, pigs, cats, dogs, and zoo animals. The methods herein for use on subjects/individuals/patients contemplate prophylactic as well as curative use. Typical subjects for treatment include persons susceptible to, suffering from or that have suffered a condition and/or disease disclosed herein.

The term “pharmaceutically acceptable carrier, excipient, or vehicle” refers to a medium which does not interfere with the effectiveness or activity of an active ingredient and which is not toxic to the hosts to which it is administered. A carrier, excipient, or vehicle includes diluents, binders, adhesives, lubricants, disintegrates, bulking agents, wetting or emulsifying agents, pH buffering agents, and miscellaneous materials such as absorbants that may be needed in order to prepare a particular composition. The use of such media and agents for an active substance is well known in the art.

The terms “preventing and/or treating”, “prevention and/or treatment”, or “prevention and/or intervention” refer to the administration to a subject of biologically active agents either before or after onset of a condition and/or disease. A treatment may be either performed in an acute or chronic way. In particular, prevention includes the management and care of a subject at risk of developing a condition and/or disease disclosed herein prior to the clinical onset of the condition and/or disease. Treatment or intervention refers to the management and care of a subject at diagnosis or later. An objective of prevention, treatment, or intervention is to combat the condition and/or disease and includes administration of the active cells or compositions disclosed herein to prevent or delay the onset of the symptoms or complications, or alleviating the symptoms or complications, or eliminating or partially eliminating the condition and/or disease.

A “beneficial effect” refers to favorable pharmacological and/or therapeutic effects, and/or improved pharmacokinetic properties and biological activity of the HipOP cell population of the present invention, or composition thereof. A beneficial effect or sustained beneficial effect may manifest as one or more of osteoblast and/or bone formation.

“Therapeutically effective amount” relates to the amount or dose of active compounds or compositions of the invention that will lead to one or more beneficial effects. A “therapeutically effective amount” can provide a dosage which is sufficient in order for prevention and/or treatment of a condition and/or disease in a subject to be effective compared with no treatment.

“Condition(s) and/or disease(s)” refers to one or more pathological symptoms or syndromes for which the cell lines disclosed herein provides a beneficial effect or therapeutic effect.

Examples of conditions and/or diseases include but are not limited to regenerative medicine, i.e., ex vivo bone engraftment in the field of orthopedic or dental surgery or a condition where bone growth or formation is beneficial.

### DETAILED DESCRIPTION OF EMBODIMENTS OF THE INVENTION

The present inventors have herein provided a method to purify and obtain a “mesenchymal stem cell” population evaluated not only by in vitro analysis but also in vivo transplantation experiments. A new population of MSC was isolated which includes a high amount of osteo-progenitor cells and has a high potential for reconstitution of skeletal tissues.

In one aspect the present invention provides for significant enrichment of a novel MSC population from murine bone marrow with high differentiation potential for osteogenesis, adipogenesis and chondrogenesis. Second, to address the in vivo differentiation potential of this population, purified murine or human cells were transplanted on collagen sponges into immunodeficient mice and found to be highly enriched in cells with a high potential for reconstitution of the skeletal system in vivo. Thus, in vitro and in vivo data presented herein indicates that this population is highly purified osteoprogenitors (HipOP) and more than OP-niche and HSCs-whole organ.

In embodiments of the invention, a pharmaceutical pack or kit is provided comprising one or more containers filled with one or more of the ingredients of a pharmaceutical composition of the invention to provide a beneficial effect, in particular a sustained beneficial effect. Associated with such container(s) can be various written materials such as instructions for use, or a notice in the form prescribed by a governmental agency regulating the labeling, manufacture, use or sale of pharmaceuticals or biological products, which notice reflects approval by the agency of manufacture, use, or sale for human administration.

In an aspect, the invention relates to a “kit-of-parts”, for example, the components to be combined according to the present invention can be dosed independently or by use of different fixed combinations with distinguished amounts of the components, i.e. simultaneously or at different time points. The parts of the kit can then be administered simultaneously or chronologically staggered, that is, at different time points and with equal or different time intervals for any part of the kit.

Parts of a kit may be administered simultaneously or chronologically staggered, i.e., at different points in time and with equal or different time intervals for any component of a kit. Time intervals can be selected such that the effect on the condition and/or disease in the combined use of the parts is larger than the effect that would be obtained by use of only any one of the components.

The invention further relates to a commercial package comprising at least one compound, and optionally an additional therapeutic agent, together with instructions for simultaneous, separate or sequential use.

In an aspect a commercial package comprising as active ingredients at least one compound is provided in the form of a separate unit, and optionally another therapeutic agent, together with instructions for its simultaneous, separate or sequential use, or any combination thereof, in the delay of progression or treatment of a condition and/or disease disclosed herein.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### EXAMPLES

### Materials and Methods

Isolation of HipOPs

Femurs of C57BL/6 mice (4-6 week-old) were harvested under sterile conditions and immersed in a-minimum essential medium (α-MEM) with antibiotics, including 100 μg/ml penicillin G (Sigma, St. Louis, Mo.), 50 μg/ml gentamycin (Sigma), and 300 ng/ml fungizone (Flow Laboratories, Mississauga, ON). After removal of the femoral heads, the marrow was collected by flushing repeatedly through the shafts with a syringe containing a-MEM supplemented with antibiotics as above and 10% heat-inactivated fetal calf serum (FCS), and sieving the cell suspension to remove call aggregates. Recovered cells were plated in a-MEM supplemented with antibiotics as above and 10% FCS. After 3 days, nonadherent calls were removed by washing 3 times with PBS. Approximately 2 weeks after seeding, when the adherent cells had expanded to cover almost 80% of the plate (subconfluent), they were detached with typsin-EDTA solution (0.2% trypsin, 1 mM EDTA). HipOPs were purified by negative sorting using anti-CD45 conjugated magnetic beads (Miltenyi Biotec, Auburn, Calif.) and a Lineage Cell Depletion Kit (Miltenyi).

For human derived cells, human bone marrow (BM) mononuclear cells, obtained by light density separation of freshly collected samples of human BM, were purchased from STEMCELL Technologies, Vancouver, Canada. Human HipOps (hHipOPs) were cultured as for murine HipOps and purified by negative selection using anti-CD45 conjugated magnetic beads and a Human Cell Lineage Depletion Kit (Miltenyi).

Flow Cytometry

Cells were blocked with purified rat anti-mouse CD16/CD32 (BD Biosciences, San Jose, Calif.) for 10 min, and then stained with respective mouse antibodies directly labeled with phycoerythrin (PE), or biotin for 15 min on ice (Table 1). APC-conjugated streptavidin (BD Biosciences) was used to reveal biotin-coupled Abs. After the staining, data from stained cells were acquired using FACSCalibur and CellQuest software (BD Biosciences).

Differentiation Assays

(a) Osteogenesis

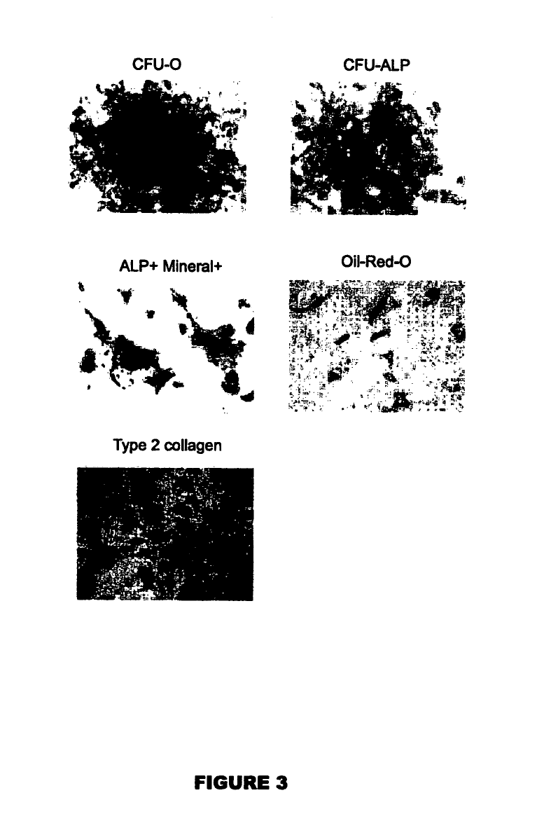

Cells were plated at various densities in 96-well plates, cultured in osteogenic induction medium comprising a-MEM, 10% FBS, antibiotics as above, 50 μg/ml ascorbic acid (Fisher Scientific Co.), 10 mM β-glycerophosphate ((β-GP) (Sigma Chemical Co.) and 10⁻⁸ M dexamethasone (Dex). After culture for 4 weeks with the medium changed every 3 or 4 days, cells were fixed in 10% neutral-buffered formalin for 30 min and double stained for alkaline phosphatase (ALP) and mineral deposition (von Kossa). After recording the presence or not of ALP- and ALP von-Kossa-positive colonies in each well, all wells were re-stained with 0.15% methylene blue for 10 min and rinsed with distilled water. Colony-forming units-osteoblast (CFU-O) were defined as colonies with ALP-positive cells associated with mineralized matrix (von-Kossa-positive) (FIG. 3). CFU-ALP colonies were defined as colonies with ALP-positive cells but no detectable mineralization (FIG. 3). Those colonies staining with methylene blue alone were defined as CFU-F; thus, the CFU-F category may contain not only cells capable of forming connective tissue, but also other progenitor types not characterized in the current experiments. Multiple discrete colonies present in the same wells were categorized and recorded separately. ALP+Mineral+were defined as small clusters of cells (1-4 cells) with mineralized matrix (FIG. 3).

(b) Adipogenesis

Cells were plated at various densities in 96-well plates, cultured in adipogenic induction medium comprising α-MEM, 10% FBS, antibiotics as above, 50 μg/ml ascorbic acid and 10⁻⁶ M BRL-49653 (a generous gift from the Sankyo Company, Tokyo), a PPAR-γ selective ligand. Medium was changed every 2 or 3 days; cells were cultured at 37° C. in a 5% CO2 humidified incubator for 1 week. Colonies with adipocytic cells (CFU-A; cells with Oil red O-positive lipid droplets) were identified by fixing in 10% neutral-buffered formalin, and staining with Oil Red 0 (a stock solution (0.5% Oil Red 0 in 100% isopropanol) was diluted 3:2 with distilled water, allowed to stand for 30 min and filtered to remove undissolved Oil Red 0 (working solution)), for 30 min, followed by rinsing with distilled water (FIG. 3).

(c) Chondrogenesis

Cells were spun down and resuspended in 1:1 DMEM/Ham F-12 (Gibco Co.), 10% FBS, antibiotics as above, 50 μg/ml ascorbic acid, 10⁻⁸ M Dex and 50 ng/ml hrBMP2 (R&D Systems, Minneapolis, Minn.). The cells were plated at various densities in 96-well plates. After culture for 2 weeks with the medium changed every 3 or 4 days, cells were fixed in 10% neutral-buffered formalin and CFU-chondrcytes (CFU-Ch) were identified by staining with goat anti-type 2 collagen antibody (Santa Cruz Biotechnology, Santa Cruz, Calif.) followed by biotinylated anti-goat IgG and a Vectastain Elite ABC kit (Vector Laboratories, Burlingame, Calif.).

Limiting Dilution

For limiting dilution analysis of osteo-, adipo-, or chondroprogenitor cell number in BMSC and HipOP populations, we used the techniques and analyses reported by the inventors earlier for rat bone marrow cells²⁶ were used. Briefly, the frequency of progenitor cells was determined by quantifying the fraction of wells not containing CFU-O, CFU-ALP, ALP+Mineral+cells, CFU-A, and CFU-Ch at each cell density tested. From a plot of the fraction of empty wells against cell number plated per well, the progenitor cell number was determined from Fo=e^{-x}, where Fo is the fraction of empty wells, and x is the mean number of osteoprogenitors per well; assuming a Poisson distribution, Fo=0.37 is the dilution at which one progenitor is present per well. In limiting dilution experiments, a minimum of 96 wells were plated for each cell density tested and the actual fraction of wells without the colony types of interest was counted and plotted ±95% confidence limits.

### Quantitative Analysis of Gene Expression

RNA was extracted with TRIzol® reagent (Invitrogen Inc., Burlington, ON) and reverse transcribed using Superscript™II (Invitrogen Inc.) according to the manufacturer's instructions. Expression levels of each lineage marker and a housekeeping gene (ribosomal protein L32) were assessed by real-time PCR (ABI Prism® 7000, Applied Biosystems, Foster City, USA) with primer sequences as listed in Table 2. The relative amounts of transcripts were normalized to the L32 transcript.

### In Vivo Transplantation

In vivo transplantation was performed as reported^{11,27}. Sterile collagen sponges (Gelfoam; Pfizer, New York, N.Y.) were cut into cubes approximately 5×5×5 mm. BMSCs or HipOPs (1.5×10⁶ cells, H-2K^{b}) were suspended in a-MEM containing 20% FCS. To load sponges with cells, the sponges were placed into the cell suspension and incubated for 90 min at 37%. The sponges were transplanted subcutaneously into 8- to 15-week old female Crlj:CD1-Foxn1^{nu} mice (H-2L^{q}).

### Immunohistochemistry

The transplants were recovered at 8 weeks after transplantation, fixed in PLP fixative (containing 4% paraformaldehyde) for 6 hr at 4° C., and decalcified with 15% EDTA for 1 week at 4° C. Decalcified transplants were frozen in ornithine carbamoyltransferase compound with liquid nitrogen. After sectioning (6 microns), H-2K^{b} positive cells were detected by staining sections with biotinylated anti-H-2K^{b} antibody and a Vectastain Elite ABC kit.

### MicroCT

A detailed qualitative and quantitative 3-D evaluation was performed of the whole transplants using a Scanco μCT40 scanner with 12 μm resolution (SCANCO Medical AG, Bassersdorf, Switzerland). A fixed threshold was applied to assess mineralized bone on the grey scale images. The total mineralized tissue volume was used for statistical analysis.

### Statistical Analysis

Values are given as means±SD of a minimum of three independent experiments, except for limiting dilution where the mean and 95% confidence limits of a minimum of 100 wells are plotted. Comparisons between means were made by using a Student's t-test. Differences between means were considered significant when P-values were less than 0.05.

### Example 1

### Characterization of New MSCs

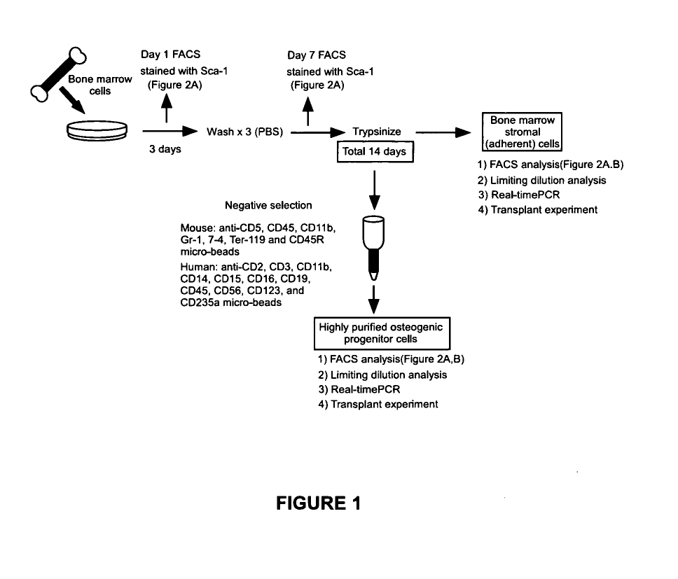

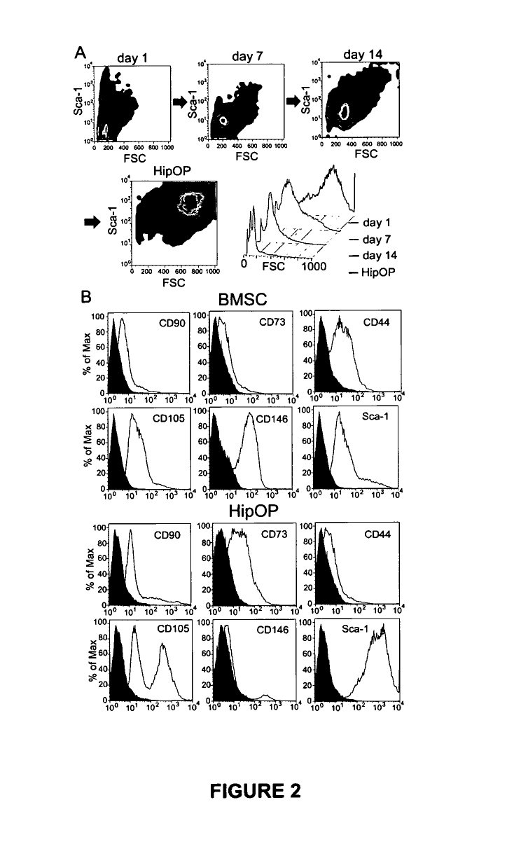

To purify an MSC-enriched population, bone marrow stromal (adherent) cells (BMSCs) were expanded up to day 14, then harvested and fractionated the populations by magnetic micro-beads (BMSCs 1.6±0.34×10⁶/mouse; HipOPs 1.1±0.37×10⁵/mouse) (FIG. 1). The size of HipOPs was quite large compared to freshly isolated and expanded BMSCs (FIG. 2a). Some cell surface markers typically considered “mesenchymal stem cell” markers were more highly expressed in HipOPs versus BMSCs (i.e., CD90, CD73 and Sca-1), while others (CD44, CD105 and CD146) were expressed at lower levels (FIG. 2b and Table 3).

FIG. 1 is a schematic diagram that illustrates the method used in the present invention to purify HipOPs. At first, bone marrow cells were plated on 10 cm dishes. The cells at 1 day after plating, including floating cells and adherent cells were used for FACS analysis as day 1 data. At 3 days after plating, dishes were washed 3 times with PBS to remove floating cells. At 7 days after plating, adherent cells were used for FACS analysis as day 7 data. Medium was changed every 3 or 4 days. At 14 days, adherent cells were harvested (BMSCs) and sorted by magnetic micro-beads (HipOPs). Both BMSCs and HipOPs were used for FACS analysis, limiting dilution experiment, real-time PCR experiment and transplant experiment. Both human and murine markers used for negative selection for the respective cell types are indicated in the figure.

FIG. 2 is the FACS analysis which reveals that HipOPs are large Sca-1+ cells. (a) BMSCs at the days indicated and sorted HipOPs were stained with APC-anti-Sca-1 Abs. Graphs are displayed with forward scatter (FSC; x-axis) and Sca-1 (y-axis). (b) BMSCs and HipOPs were stained with the panel of typical “mesenchymal stem cell markers”. This figure is using murine derived cells.

### Example 2

### Evaluation of HipOps' Differentiation Ability to Osteoblast, Adipocyte and Chondrocyte

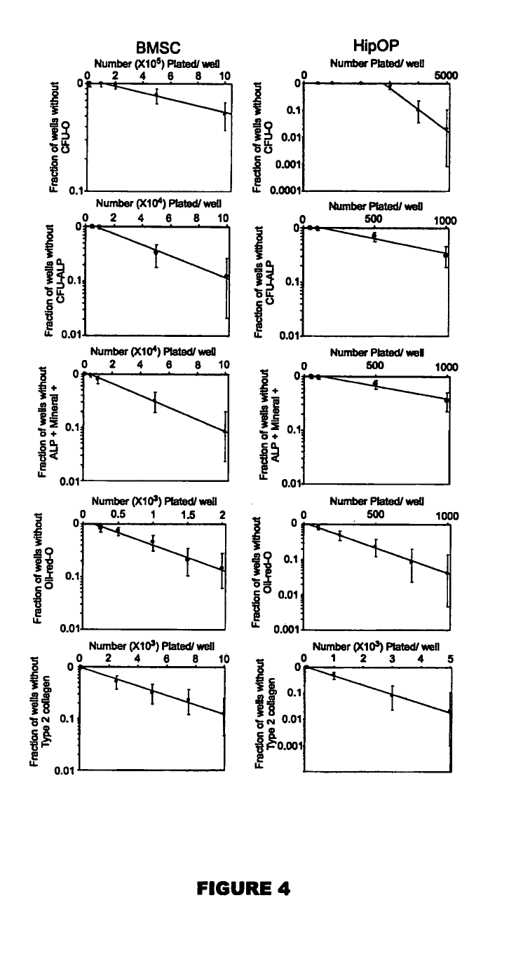

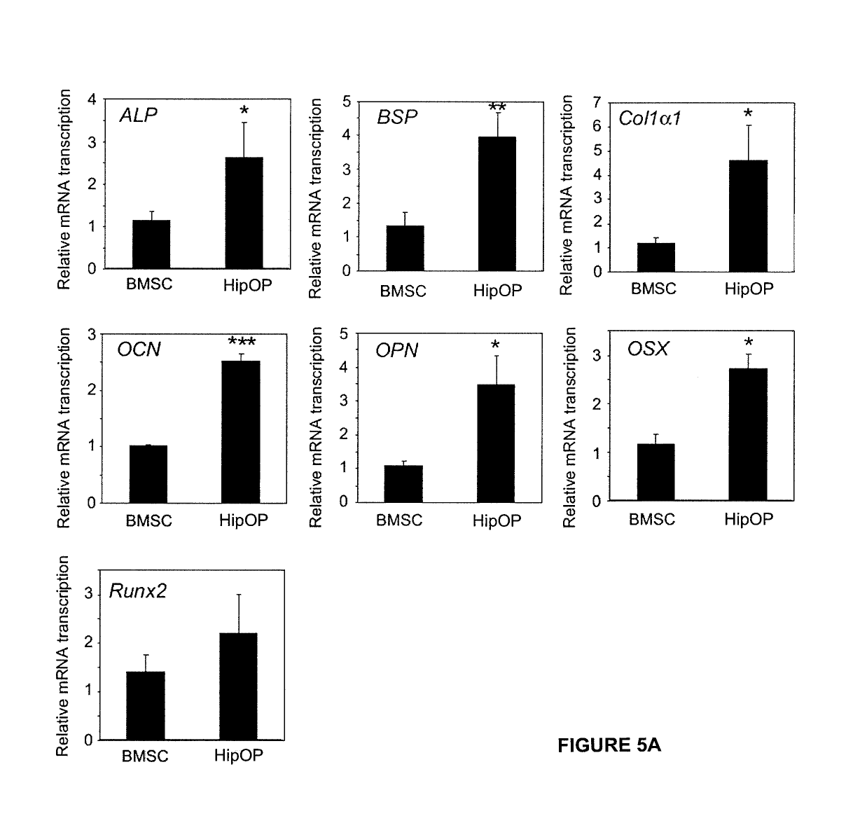

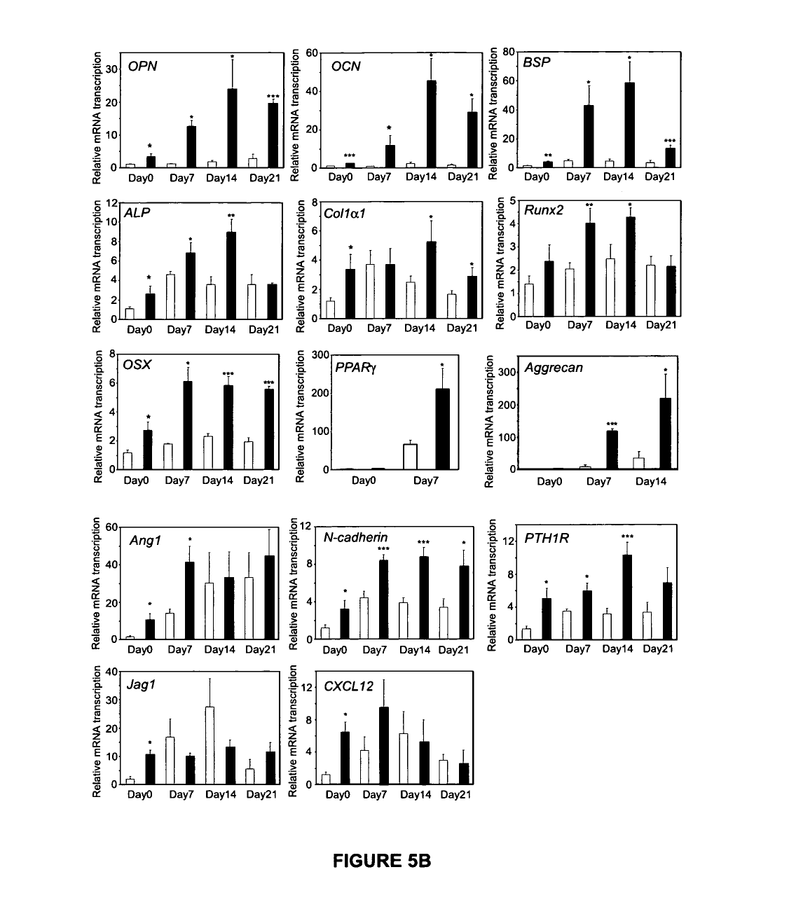

Since MSCs are expected to have capacity for differentiation along multiple mesenchymal lineages, HipOPs were cultured under conditions supportive of osteoblastic, adipocytic or chondrocytic development and differentiation confirmed along all three lineages (FIG. 3). To confirm that the frequency of MSCs was increased in murine HipOP versus murine BMSC populations, limiting dilution analyses were performed. The frequency of CFU-O was 100 times higher in HipOP than in BMSC cells (FIG. 4 and Table 4). Also increased was the frequency of CFU-ALP and the frequency of individual or small clusters of cells (1-3 cells; not counted as a CFU) which are ALP-positive and have associated mineral deposits (almost 10 times and 60 times, respectively) (FIG. 4 and Table 4). The adipocyte and chondrocyte (CFU-Ch) progenitor frequency were also higher in HipOP versus BMSCs (almost 2 times and 5 times, respectively) (FIG. 4 and Table 4). Consistent with colony counts, the expression levels of differentiation markers for all three lineages (for osteoblasts: OPN, OCN, BSP and OSX); for PPARg for adipocytes; aggrecan for chondrocytes) were higher in HipOPs than BMSCs (FIGS. 5A and 5B). Thus, both limiting dilution and real-time PCR analyses demonstrated that the HipOPs is highly enriched in multipotential MSCs and have a high differentiation capacity for osteoblasts.

FIG. 3 illustrates the results of (CFU-O, CFU-ALP and ALP+Mineral+) BMADs or HipOPs cultured in osteogenic induction medium. After culture for 4 weeks, cells were double stained for ALP and mineral deposition (von Kossa), and were re-stained with methylene blue. (Oil-Red-O) Cells were cultured in adipogenic induction medium 1 week. Adipocyte colonies were identified by Oil Red O staining. (Type 2 collagen) Cells were cultured in chondrogenic induction medium for 2 weeks. Cells were stained with anti-type 2 collagen antibody and a Vectastain Elite ABC kit, and re-stained with methylene blue.

FIG. 4 illustrates that limiting dilution analysis shows that multiple mesenchymal progenitor types are enriched in the HipOP as compared to the BMSCs. Cells were plated at the densities specified into 96-well microtiter trays, and the frequency of wells without colonies of each type was quantified. Values are the mean and 95% confidence limits.

FIG. 5A illustrates the comparison of osteoblast differentiation markers between BMSCs and HipOPs at day 0. mRNA was extracted from harvested BMSCs and sorted HipOPs. mRNA was reverse transcribed in three independent experiments. Samples were subjected to quantitative real-time PCRs using specific primers for OPN, OCN, BSP, ALP, Type I collagen, Runx2 and OSX. mRNA expression were normalized to L32 expression. Values are expressed as means±S.D. Asterisks indicate statistically significant differences: p<0.05 (*), p<0.01 (**), p<0.005 (***).

FIG. 5B HipOPs show higher expression of mesenchymal cell differentiation marker genes. BMSCs and HipOPs were cultured with osteogenic induction medium, adipogenic induction medium, or chondrogenic induction medium. mRNA was extracted and reverse transcribed in three independent experiments. Samples were subjected to quantitative real-time PCRs using specific primers for OPN, OCN, BSP, ALP, Type I collagen, Runx2, OSX, PPARy, Aggrecan, Ang1, N-cadherin, PTH1R, Jag1, and CXCL12, RNA expression were normalized to L32 expression. Values are expressed as means±S.D. Asterisks indicate statistically significant differences: p<0.05 (*), p<0.01 (**), p<0.005 (***).

### Example 3

### Transplantation Experiment of HipOps

### Mouse:

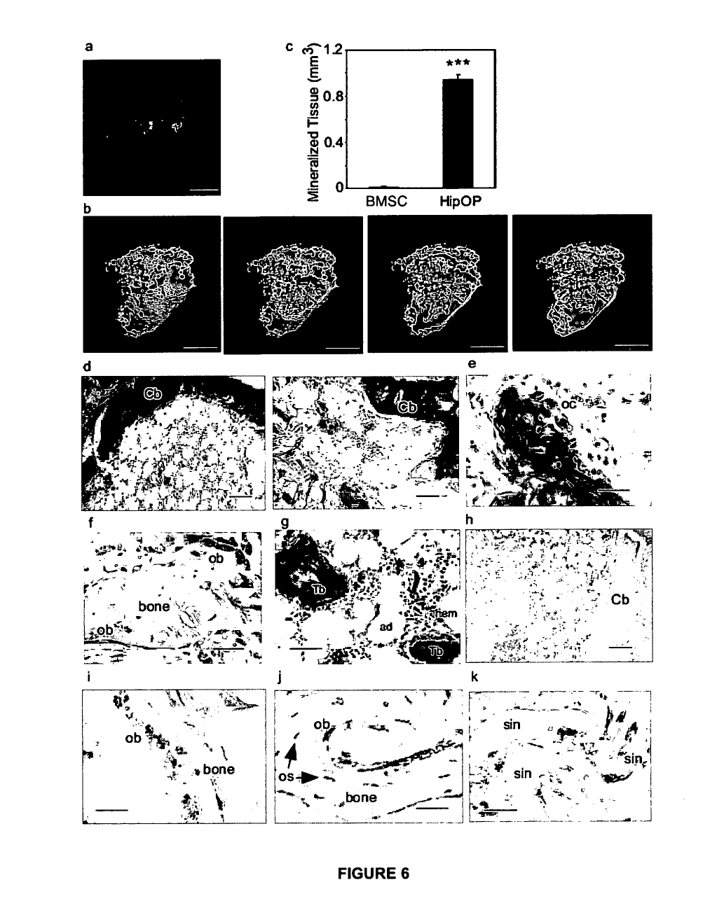

Given the robust osteoblast differentiation capacity of HipOP cells in vitro, it was then investigated as to whether HipOPs showed enhanced differentiation capacity for skeletal tissue in vivo. When 1.5×10⁶ cells BMSCs or HipOPs in collagen sponges were transplanted subcutaneously into Crlj:CD1-Foxn1^{nu} mice, and analyzed at 8 weeks, both macroscopic observation (not shown) and microCT revealed only a few small areas of mineralization in transplants with BMSCs (FIG. 6a), but large masses of mineralized tissue (over 100 times higher than those seen in BMSCs) were seen in transplants with HipOP cells (FIG. 6b,c). Histologial sections confirmed that HipOPs formed complex skeletal structures, with areas of cortical bone, trabecular bone, adipose tissue, osteoblasts, osteoclasts, and vascular structures (FIG. 6d-g). HLA typing with H-2K^{b} antibodies showed that donor cells were present throughout the structures, with H-2K^{b}-positive osteoblasts, osteocytes and cells around sinusoids (FIG. 6h-k); H-2K^{b}-positive bone marrow cells were also present (FIG. 6h-k).

FIG. 6 HipOPs form a complete skeletal organ after transplantation. (a) MicroCT 3-D reconstructions of a typical transplant of BMSCs at 8 weeks after transplantation. Bar=1 mm. (b) MicroCT 3-D reconstructions of a typical transplant of HipOPs at 8 weeks after transplantation. Bar=1 mm. (c) The comparison of volume of total mineralized tissue between BMSCs and HipOPs. Data are expressed as means of three independent experiments±S.D. Asterisks indicate statistically significant differences: p<0.005 (***). (d-g) Histology of transplants of HipOPs harvested at 8 weeks. Cb, cortical bone; Tb, trabecular bone; ob, osteoblast; oc, osteoclast; ad, adipose tissue; hem, hematopoietic cells, H&E. d ×100: e-g ×400. Bars=50 μm (d): Bars=30 μm (e-g). (h-k) Frozen sections of transplants of HipOPs) were stained with anti-H-2K^{b} Abs and re-stained with fast green. sin, sinusoid; os, osteocyte. h ×100: i-k ×400. Bars=50 μm (h): Bars=30 μm (i-k).

### Human:

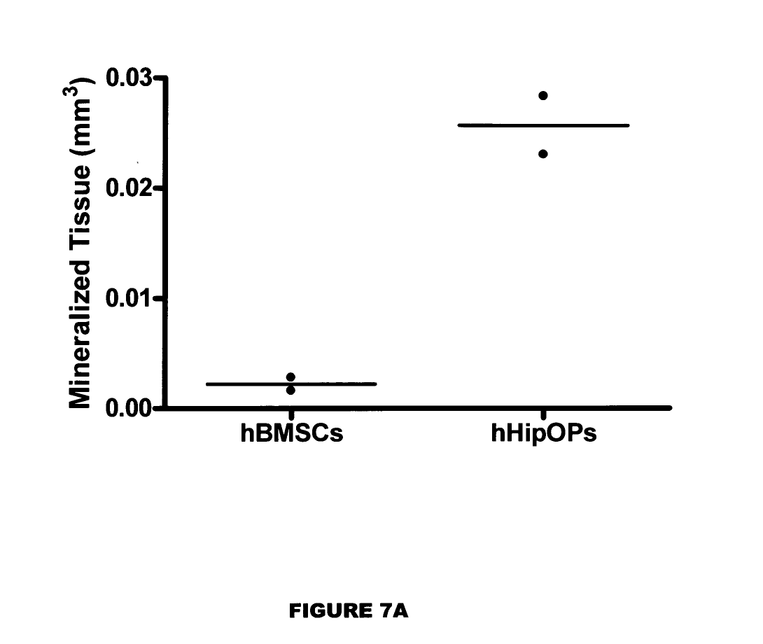

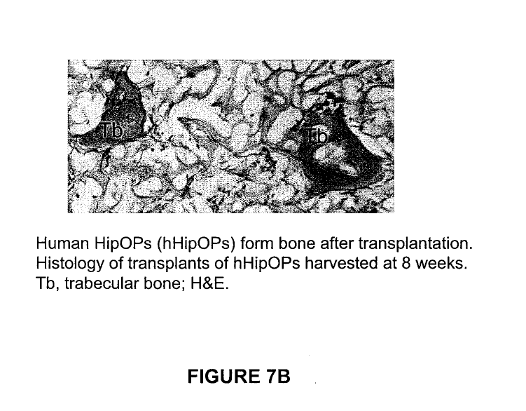

The same experiments as illustrated for the mouse in FIG. 6 was performed with human cells. FIG. 7A is a graph illustrating the amount of bone formed in transplants seeded with unfractionated human bone marrow stromal cells (hBMSCs) or with human HipOPs as a comparison of volume of total mineralized tissue between hBMSCs and hHipOPs. Data are from two independent experiments; the symbols indicate the values from individual experiments and the line marks the average of the two values. Approximately 10× more bone was formed in transplants seeded with hHipOPs than in transplants seeded with hBMSCs. Histologial sections confirmed that HipOPs formed complex skeletal structures, with areas of trabecular bone (Tb) (FIG. 7B)

### Discussion

Surface phenotypic characteristics of cells designated MSCs differ among laboratories and species, and there is no one specific marker or combination of markers that unambiguously identify MSCs either in vivo or in vitro.

Phenotypically, it is generally accepted that MSCs variably express CD90 (Thy1.1), CD117 (c-kit), SH2 (CD105 or endoglin), SH3 or SH4 (CD73)¹⁷⁻¹⁹. Both the unsorted BMSCs and HipOPs express CD90, CD105, CD73 and CD44, with CD90- and CD73-positive cells significantly enriched and CD105- and CD44-positive cells significantly decreased in the HipOP versus BMSC population. MSCs are typically thought to be positive for MHC I and Sca-1^{17 20 21, 22}. An interesting recent study in a Sca-1/Ly-6A null mouse documented normal skeletal development with age-dependent development of osteoporosis associated with a defect in mesenchymal progenitor self-renewal²³. The present examples indicate that essentially 100% of HipOP cells are Sca-1-positive which indicates that Sca-1 is related to the maintenance of MSCs and may be a significant stem cell marker not only on the surface of hematopoietic stem cells (HSCs) but also MSCs. It should also be noted that the method of the present invention does not exclude CD34-positive cells from the HipOP fraction. Although human and rat MSCs have been reported to be negative for expression of CD34, murine MSCs have variously been described to express or not CD34 expression, with acquisition of the marker in vitro^{20 21 22}. In the present experiments, the same percent BMSCs and HipOPs express CD34 (BMSCs 17.3±1.2%; HipOPs 17.6±2.5%). It is possible that some markers of MSCs overlap with those of HSCs like Sca-1. In any case, the present experiments indicated that CD34 may be a useful candidate for positive selection of HipOPs. Recently, Bianco et al demonstrated the utility of CD146 as a marker for those self-renewing bone marrow stromal cells/CFU-Fs with a critical role for reconstitution of both osteoblasts making bone and those creating the hematopoietic microenviroment²⁴. In contrast, the present inventors found a high percent of cells in the unfractionated murine BMSC expressed CD146, but far fewer in the HipOP fraction which was, on the other hand, enriched for all mesenchymal progenitor types including osteoprogenitors and the cells expressing markers of the niche for HSCs. That expression level of surface markers is sometimes quite different between human and murine cells is already well-established²⁵, and the data herein suggest that an alternative marker(s) for CD146 may characterize the murine MSC and HSC niche population.

Limiting dilution analyses demonstrated that HipOPs are enriched for cells with multilineage differentiation capacity including capacity to generate osteoblasts, adipocytes and chondrocytes. However, amongst these, CFU-O's and small clusters (single cells or small groups of 2-4 cells) of ALP+Mineral+ cells were most abundant, at over 100 times and 50 times higher frequency in BMSCs and

HipOPs respectively. Thus, HipOPs have high capacity for osteogenic differentiation. These results were confirmed by in vivo transplantation experiment. The total mineral volume of murine HipOPs is 100 times higher than BMSCs (BMSCs 0.0092±0.0089 mm³; HipOPs 0.94±0.043 mm³). The total mineral volume of human HipOPs is also 10 times higher than hBMSCs (hBMSCs 0.0022±0.0085 mm³; hHipOPs 0.026±0.0037 mm³) MicroCT 3-D data clearly showed that HipOPs can reconstruct the bone structure in the transplant. The mass of HipOPs' transplant was covered with cortical bone and has the cavity inside. The inside cavity has a lot of trabecular bones, adipose tissues and vascular system. The cortical bone surface has a lot of holes which might be the way of vascular systems to supply blood stream into inside cavity. The H-2K^{b} positive cells surround sinusoids which indicates HipOPs might support the establishment of vascular system in the transplant.

Recently, several groups proposed models of HSC niche; 1) Tie2/Angiopoietin1 (Ang1) signalling between HSCs and osteoblasts enhances N-cadherin-mediated adhesion, and then contribute to HSCs quiescence, 2)

Parathyroid hormone 1 receptor (PTH1R) is not only known to induce osteoblast differentiation but also recently known that osteoblasts activated through PTH1R induce an increase of the Notch ligand jagged1 (Jag1) on its surface and then support an increase of the number of HSCs in bone marrow niche, 3) CXCL12-CXCR4 signaling (osteoblasts-HSCs) is an important role for the maintenance of HSCs pool. To address whether HipOPs contain component cells of HSC niche, expression levels of HSC niche marker genes i.e., Ang1, N-cadherin, PTH1R, Jag1, and CXCL12 were analysed. The expression levels of all HSC niche marker genes in HipOPs were higher than in BMSCs at day 0 (Ang1; 5 times, N-cadherin; 3 times, PTH1R; 3 times, Jag1; 5 times, CXCL12; 5 times, respectively) (FIG. 5B). The results indicate that HipOPs include large component of cells for HSC niche. The high expression levels of N-cadherin and PTH1R were observed until day 14 or day 21 in HipOPs (FIG. 5B). This observation reflects the high osteogenic activity of HipOPs.

In summary, the inventors have succeeded in purifying a novel bone marrow-derived population that manifests robust enrichment for MSC-like cells, for cells with high potential for reconstitution of a bone organ structure in vivo, and for cells associated with the HSC niche. In one aspect, the novel cell population of the invention can be used in regenerative medicine, i.e., ex vivo bone engraftment in the field of orthopaedic or dental surgery or in conditions where bone formation is beneficial. In one embodiment, the cells can be used in conditions where bone growth would be beneficial.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein, or referenced in such documents are incorporated by reference in their entirety to the same extent as if each individual publication, patent or patent application was specifically and individually indicated to be incorporated by reference in its entirety. The citation of any reference herein is not an admission that such reference is available as prior art to the instant invention.

TABLE 1
Antibodies used for FACS

| Antigen | Labeling | Clone | Distributor |
|---|---|---|---|
| Sca-1 | Biotin | D7 | BD Biosciences |
| CD34 | Biotin | MEC14.7 | Biolegend |
| CD44 | APC | IM7 | BD Biosciences |
| CD73 | PE | TY/23 | BD Biosciences |
| CD90 | Biotin | 53-2.1 | BD Biosciences |
| CD105 | Biotin | MJ7/18 | Biolegend |
| CD146 | PE | P1H12 | Biolegend |

TABLE 2
Primers for Real-time PCR [NOTE: Confirm these
are known primers] yes-we have published
previously

| | | SEQ | |
|---|---|---|---|
| | Accession | ID | |
| Gene | number | NO. | Sequence |
| L32- | NM_172086 | 1 | CAC AAT GTC AAG GAG CTG GAA |
| Forward | | | GT |
| L32- | | 2 | TCT ACA ATG GCT TTT CGG TTC |
| Reverse | | | T |
| OPN- | NM_009263 | 3 | AGC AAG AAA CTC TTC CAA GCA |
| Forward | | | A |
| OPN- | | 4 | GTG AGA TTC GTC AGA TTC ATC |
| Reverse | | | CG |
| OCN- | NM_007541 | 5 | CTG ACC TCA CAG ATG CCA AGC |
| Forward | | | |
| OCN- | | 6 | TGG TCT GAT AGC TCG TCA CAA |
| Reverse | | | G |
| BSP- | NM_008318 | 7 | CAG GGA GGC AGT GAC TCT TC |
| Forward | | | |
| BSP- | | 8 | AGT GTG GAA AGT GTG GCG TT |
| Reverse | | | |
| ALP- | NM_007431 | 9 | CCA ACT CTT TTG TGC CAG AGA |
| Forward | | | |
| ALP- | | 10 | GGC TAC ATT GGT GTT GAG CTT |
| Reverse | | | TT |
| Collαl- | NM_007742 | 11 | GCT CCT CTT AGG GGC CAC T |
| Forward | | | |
| Collαl- | | 12 | CCA CGT CTC ACC ATT GGG G |
| Reverse | | | |
| Runx2- | NM_009820 | 13 | TGT TCT CTG ATC GCC TCA GTG |
| Forward | | | |
| Runx2- | | 14 | CCT GGG ATC TGT AAT CTG ACT |
| Reverse | | | CT |
| OSX- | NM_130458 | 15 | ATG GCG TCC TCT CTG CTT G |
| Forward | | | |
| OSX- | | 16 | TGA AAG GTC AGC GTA TGG CTT |
| Reverse | | | |
| PPARγ- | NM_011146 | 17 | TGA AAC TCT GGG AGA TTC TCC |
| Forward | | | TG |
| PPARγ- | | 18 | CCA TGG TAA TTT CTT GTG AAG |
| Reverse | | | TGC |
| Aggrecan- | NM_007424 | 19 | GCG TGA GCA TCC CTC AAC CAT |
| Forward | | | C |
| Aggrecan- | | 20 | GGC AGT GGT CAC AGG ATG CAT |
| Reverse | | | G |

TABLE 3
Flow cytometry analysis of BMSCs and HipOPs

Positive Cells (%)

| | BMSCs | HipOPs |
|---|---|---|
| CD90 | 20.3 ± 2.4 | 26.4 ± 2.3* |
| CD73 | 10.9 ± 0.7 | 43.0 ± 1.3*** |
| CD44 | 92.8 ± 2.3 | 36.9 ± 3.1*** |
| CD105 | 84.2 ± 2.2 | 62.7 ± 5.9*** |
| CD146 | 81.1 ± 1.2 | 10.2 ± 1.8** |
| Sca-1 | 79.2 ± 2.7 | 98.3 ± 0.5*** |

Values are the means ± S.D. of three independent experiments. Asterisks indicate statistically significant differences: p < 0.05 (*), p < 0.01 (**), p < 0.005 (***).

TABLE 4
Summary of limiting dilution analysis

| | |
|---|---|
| BMADs | HipOPs |

CFU-ALP

| | | |
|---|---|---|
| #1 | 1/6,700 | 1/280 |
| #2 | 1/7,800 | 1/580 |
| #3 | 1/7,500 | 1/780 |

ALP + mineral+

| | | |
|---|---|---|
| #1 | 1/42,000 | 1/260 |
| #2 | 1/42,000 | 1/730 |
| #3 | 1/43,000 | 1/880 |

CFU-O

| | | |
|---|---|---|
| #1 | 1/130,000 | 1/740 |
| #2 | 1/140,000 | 1/820 |
| #3 | 1/150,000 | 1/1,000 |

CFU-F

| | | |
|---|---|---|
| #1 | 1/1,800 | 1/340 |
| #2 | 1/1,900 | 1/340 |
| #3 | 1/2,900 | 1/460 |

CFU-A

| | | |
|---|---|---|
| #1 | 1/700 | 1/300 |
| #2 | 1/700 | 1/300 |
| #3 | 1/1,000 | 1/400 |

CFU-Ch Type 2 collagen

| | | |
|---|---|---|
| #1 | 1/5,000 | 1/1,000 |
| #2 | 1/5,400 | 1/1,000 |
| #3 | 1/5,700 | 1/1,200 |

See Materials and Methods for definitions and identification protocols for all colony types

### REFERENCES CITED

1. Colter, D. C., Sekiya, I. & Prockop, D. J. Identification of a subpopulation of rapidly self-renewing and multipotential adult stem cells in colonies of human marrow stromal cells. Proc Natl Acad Sci USA 98, 7841-7845 (2001).
2. Digirolamo, C. M. et al. Propagation and senescence of human marrow stromal cells in culture: a simple colony-forming assay identifies samples with the greatest potential to propagate and differentiate. Br J Haematol 107, 275-281 (1999).
3. Friedenstein, A. J., Gorskaja, J. F. & Kulagina, N. N. Fibroblast precursors in normal and irradiated mouse hematopoietic organs. Exp Hematol 4, 267-274 (1976).
4. Jaiswal, N., Haynesworth, S. E., Caplan, A. I. & Bruder, S. P. Osteogenic differentiation of purified, culture-expanded human mesenchymal stem cells in vitro. J Cell Biochem 64, 295-312 (1997).
5. Owen, M. & Friedenstein, A. J. Stromal stem cells: marrow-derived osteogenic precursors. Ciba Found Symp 136, 42-60 (1988).
6. Owen, M. Marrow stromal stem cells. J Cell Sci Suppl 10, 63-76 (1988).
7. Phinney, D. G., Kopen, G., Isaacson, R. L. & Prockop, D. J. Plastic adherent stromal cells from the bone marrow of commonly used strains of inbred mice: variations in yield, growth, and differentiation. J Cell Biochem 72, 570-585 (1999).
8. Piersma, A. H. et al. Characterization of fibroblastic stromal cells from murine bone marrow. Exp Hematol 13, 237-243 (1985).
9. Prockop, D. J. Marrow stromal cells as stem cells for nonhematopoietic tissues. Science 276, 71-74 (1997).
10. Wakitani, S., Saito, T. & Caplan, A. I. Myogenic cells derived from rat bone marrow mesenchymal stem cells exposed to 5-azacytidine. Muscle Nerve 18, 1417-1426 (1995).
11. Bianco, P., Kuznetsov, S. A., Riminucci, M. & Gehron Robey, P. Postnatal skeletal stem cells. Methods Enzymol 419, 117-148 (2006).
12. Kerk, D. K., Henry, E. A., Eaves, A. C. & Eaves, C. J. Two classes of primitive pluripotent hemopoietic progenitor cells: separation by adherence. J Cell Physiol 125, 127-134 (1985).
13. Bearpark, A. D. & Gordon, M. Y. Adhesive properties distinguish sub-populations of haemopoietic stem cells with different spleen colony-forming and marrow repopulating capacities. Bone Marrow Transplant 4, 625-628 (1989).
14. Simmons, P. J. et al. Vascular cell adhesion molecule-1 expressed by bone marrow stromal cells mediates the binding of hematopoietic progenitor cells. Blood 80, 388-395 (1992).
15. Deryugina, E. I. & Muller-Sieburg, C. E. Stromal cells in long-term cultures: keys to the elucidation of hematopoietic development? Crit Rev Immunol 13, 115-150 (1993).
16. Witte, P. L. et al. Relationships between B-lineage lymphocytes and stromal cells in long-term bone marrow cultures. Eur J Immunol 17, 1473-1484 (1987).
17. Pittenger, M. F. et al. Multilineage potential of adult human mesenchymal stem cells. Science 284, 143-147 (1999).
18. Haynesworth, S. E., Baber, M. A. & Caplan, A. I. Cell surface antigens on human marrow-derived mesenchymal cells are detected by monoclonal antibodies. Bone 13, 69-80 (1992).
19. Chamberlain, G., Fox, J., Ashton, B. & Middleton, J. Concise review: mesenchymal stem cells: their phenotype, differentiation capacity, immunological features, and potential for homing. Stem Cells 25, 2739-2749 (2007).
20. Colter, D. C., Class, R., DiGirolamo, C. M. & Prockop, D. J. Rapid expansion of recycling stem cells in cultures of plastic-adherent cells from human bone marrow. Proc Natl Acad Sci USA 97, 3213-3218 (2000).
21. Javazon, E. H., Colter, D. C., Schwarz, E. J. & Prockop, D. J. Rat marrow stromal cells are more sensitive to plating density and expand more rapidly from single-cell-derived colonies than human marrow stromal cells. Stem Cells 19, 219-225 (2001).
22. Peister, A. et al. Adult stem cells from bone marrow (MSCs) isolated from different strains of inbred mice vary in surface epitopes, rates of proliferation, and differentiation potential. Blood 103, 1662-1668 (2004).
23. Bonyadi, M. et al. Mesenchymal progenitor self-renewal deficiency leads to age-dependent osteoporosis in Sca-1/Ly-6A null mice. Proc Natl Acad Sci USA 100, 5840-5845 (2003).
24. Sacchetti, B. et al. Self-renewing osteoprogenitors in bone marrow sinusoids can organize a hematopoietic microenvironment. Cell 131, 324-336 (2007).
25. Javazon, E. H., Beggs, K. J. & Flake, A. W. Mesenchymal stem cells: paradoxes of passaging. Exp Hematol 32, 414-425 (2004).
26. Aubin, J. E. Osteoprogenitor cell frequency in rat bone marrow stromal populations: role for heterotypic cell-cell interactions in osteoblast differentiation. J Cell Biochem 72, 396-410 (1999).
27. Krebsbach, P. H. et al. Bone formation in vivo: comparison of osteogenesis by transplanted mouse and human marrow stromal fibroblasts. Transplantation 63, 1059-1069 (1997).

1. A method for purifying mesenchymal cells comprising the steps of:
(a) providing or obtaining bone marrow cells from a mouse or human;
(b) expanding the cells;
(c) negatively selecting cells for the following cell markers: CD5, CD45, CD11b, Gr-1, 7-4, Ter-119 and CD45 when the cells are derived from a mouse, and CD2, CD3, CD11b, CD14, CD15, CD16, C19, CD45, CD56, CD123, and CD235a when the cells are derived from a human;
(d) collecting the negatively selected for cells.

2. The method for claim 1, wherein in the negative selection of step (c) comprises fractionating the cells using marker conjugated microbeads selected from one or more of the following markers: CD5, CD45, CD11b, Gr-1, 7-4, Ter-119 and CD45 when the cells are derived from a mouse, and CD2, CD3, CD11b, CD14, CD15, CD16, C19, CD45, CD56, CD123, and CD235a when the cells are derived from a human and the collection of step (d) comprises collecting the fraction of mesenchymal progenitor cells that did not adhere to said microbeads.

3. The method of claim 1, comprising treating the cells prior to and/or after expansion to obtain a cell suspension.

4. The method of claim 3, further comprising removing cellular aggregates from the cell suspension.

5. The method of claim 1, wherein the cells were expanded for about 14 days.

6. The method of claim 1, wherein the cells were expanded to subconfluent.

7. The method of claim 1, wherein CD34+ cells are not negatively selected for.

8. A method of reconstituting bone or forming bone structure or osteoblasts in a subject comprising transplanting the cells isolated from claim 1 into said subject.

9. A mesenchymal cell population isolated using the method of claim 1.

10. A mesenchymal cell population of claim 9 that comprises CD34+ cells and Sca-1+ cells.

11. A mesenchymal cell population of claim 10 that can differentiate into osteoblasts, adipocytes and chondrocytes under appropriate culture conditions.

12. A mesenchymal cell population of claim 11 wherein the culture conditions include culturing the cells in an appropriate inducing medium.

13. A mesenchymal cell population of claim 11 that can form bone structure.

14. A mesenchymal cell population of claim 11 which can form bone when transplanted into a subject.

15. A composition comprising a mesenchymal cell population of claim 11.

16. A method for treating a condition where bone formation is beneficial comprising transplanting the population of cells of claim 1 to a subject in need thereof under conditions that induce bone and/or osteoblast formation in said subject.

17. A method for treating a condition where bone formation is beneficial comprising transplanting the population of cells of claim 11 to a subject in need thereof under conditions that induce bone and/or osteoblast formation in said subject.

18. An isolated murine mesenchymal cell population that is negatively selected for the following markers: CD5, CD45, CD11b, Gr-1, 7-4, Ter-119 and CD45.

19. The isolated murine mesenchymal cell population of claim 18 that is positive for one or more of Sca-1, CD34, CD73 and CD90.

20. An isolated human mesenchymal cell population that is negatively selected for the following markers: CD2, CD3, CD11b, CD14, CD15, CD16, C19, CD45, CD56, CD123, and CD235a and optionally positive for one or more of Sca-1, CD34, CD73 and CD90.
